(19) United States
(12) Patent Application Publication
(10) Pub. No.: US 2007/0185520 A1
(43) Pub. Date: Aug. 9, 2007

(54) Detachable medical immobilization device and related methods of use

(75) Inventors: Eric Cheng, Bloomington, IN (US); Amy Foust, Bloomington, IN (US)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: BOSTON SCIENTIFIC SCIMED, Inc.

(21) Appl. No.: 11/348,311

(22) Filed: Feb. 7, 2006

(51) Int. Cl. A61M 29/00

(52) U.S. Cl. 606/191

(57) ABSTRACT

Embodiments of the invention are directed to a medical device and methods for immobilizing and retrieving material from a patient's body. The medical device includes an expansible member having a proximal end, a delivery state, and an expanded, deployed state. An instrument is detachably engaged with the proximal end of the expansible member to deploy the expansible member within an anatomical lumen beyond material to be retrieved. The expansible member in the expanded state is configured to inhibit movement of the material within the anatomical lumen.

### FIELD OF THE INVENTION

This invention relates to medical devices and methods for retrieving objects within anatomical lumens of the body. More particularly, the invention relates to methods, and devices, for retrieving and preventing undesired migration of material, such as urinary tract stones, gall stones, and other objects within anatomical lumens of the body, during a medical procedure.

### BACKGROUND OF THE INVENTION

Medical immobilization and retrieval devices may include devices for stabilizing and/or removing organic material (e.g., blood clots, tissue, and biological concretions such as urinary, biliary, and pancreatic stones) and inorganic material (e.g., components of a medical device or other foreign matter), which may obstruct or otherwise be present within a body's anatomical lumens. For example, concretions can develop in certain parts of the body, such as in the kidneys, pancreas, and gallbladder. Minimally invasive medical procedures generally involve causing limited trauma to the tissues of a patient, and can be used to dispose of problematic concretions. Lithotripsy and ureteroscopy, for example, are used to treat urinary calculi (e.g., kidney stones) in the ureter of a patient.

Lithotripsy is a medical procedure that uses energy in various forms such as acoustic shock waves, pneumatic pulsation, electrical hydraulic shock waves, or laser beams to break up biological concretions such as urinary calculi (e.g., kidney stones). The force of the energy, when applied either extracorporeally or intracorporeally, usually in focused and continuous or successive bursts, divides a kidney stone into smaller fragments that may be extracted from the body or allowed to pass through urination.

When stones are fragmented within a body tract by a lithotriptor, the stone must first be stabilized. Typically, a medical retrieval device, such as a surgical grasper or a metal wire basket, is used to capture a stone in the retrieval assembly. With the stone held in position within the retrieval assembly, a lithotriptor, such as a laser lithotriptor, comes into proximity with the stone and the stone is fragmented by the lithotriptor. After the stone is fragmented, the stone fragments can be removed by the same or a different medical retrieval device, or the fragments can be left in the body to be eliminated naturally. With the help of imaging tools such as transureteroscopic video technology and fluoroscopic imaging, the operator of the lithotripter device can monitor the progress of the medical procedure and terminate treatment when residual fragments are small enough to be voided or removed.

Intracorporeal fragmentation of urinary calculi can prove problematic in that stones and/or stone fragments in the ureter may become repositioned closer to and possibly migrate back toward the kidney, thereby requiring further medical intervention to prevent the aggravation of the patient's condition. Existing practices to control migration of stones during lithotripsy include reducing the energy or frequency of the lithotripsy, or reducing the amount or frequency of irrigation used during the procedure. Another known practice includes pushing the stone into the renal pelvis and undertaking another future procedure for its removal.

Various devices may be deployed to control migration and aid in retrieval of fragmented stones. For example, combined immobilization and retrieval devices may be deployed within a patient's body, independently, or through the working channel of an endoscope. Once deployed past the stone, the immobilization device can act as a backstop to prevent upward migration of fragments resulting from a lithotripsy procedure.

Laser lithotriptors, for example, are effective in fragmenting stones that are captured in a retrieval assembly of a medical retrieval device. One drawback of the combined use of a laser lithotriptor and a backstop and/or retrieval assembly is the susceptibility of the assembly, or parts of the assembly, to laser energy-induced damage. Damage may be caused by misfiring, misdirection, or unavoidable misalignment of the laser lithotriptor with the stone. Laser energy-induced retrieval assembly damage may cause components of the backstop and/or retrieval assembly, such as portions of a traditional metal basket, to become roughened or broken. Broken or roughened portions of the device expose sharp ends or surfaces that can traumatize the delicate internal lining of the ureter. In addition, further damage to the patient can occur where misdirected laser energy is improperly absorbed by the retrieval device, thereby heating the retrieval device (and patient tissue) to unintended dangerous levels.

Known medical devices for preventing the migration of stones and fragments are often deployed beyond a stone in a configuration that partially occludes the lumen or acts as a barrier to prevent the passage of unwanted material beyond a treatment site. The occluding elements are often made of materials formed at least partially of shape-memory materials, such as, stainless steel, nitinol, copper, cobalt, vanadium, chromium, iron, or the like. The continued deployment, repositioning, and movement of these metallic materials within a patient's body lumen can often cause undesired irritation and unnecessary trauma to the patient's body tract.

Another drawback of the combined use of a laser lithotriptor and a backstop and/or retrieval assembly is the restraint on movement imposed by the relatively small anatomical working areas accessed for treatment. Known backstop and retrieval devices extend proximally from a treatment site to a point outside of the patient where they are controllably positioned by an operator. Therefore, in embodiments where a backstop is deployed distally beyond a kidney stone, the proximal portion of the backstop (which necessarily extends outside the patient) congests and obstructs the free movement of an associated lithotriptor device. Furthermore, congestion is further exacerbated where the treatment procedure requires the use of an endoscope at the working area for lighting and imaging the treatment area.

The resulting congestion at the treatment site hinders the free movement of various components placed at the working area during a treatment procedure. This restraint on movement can lead to misfiring, misdirection, or unavoidable misalignment of the laser lithotriptor with a kidney stone, for example, thereby prolonging treatment and in some cases causing injury to a patient's tissues.

Thus, it is desirable to have alternative methods and devices for preventing upward migration of fragments, and extracting such fragments while limiting trauma to the patient.

### SUMMARY OF THE INVENTION

Embodiments of the present invention are directed to medical devices for immobilization and/or retrieval of objects within anatomical lumens of the body that obviate one or more of the limitations and disadvantages of prior immobilization and retrieval devices.

In one embodiment, the medical device includes an expansible member having a proximal end, a delivery state, and an expanded, deployed state. An instrument is detachably engaged with the proximal end of the expansible member to deploy the expansible member within an anatomical lumen beyond material to be retrieved. The expansible member in the expanded state is configured to inhibit movement of the material within the anatomical lumen.

In various embodiments, the medical device may include one or more of the following additional features: an elongated flexible tube including a distal end and a proximal end, the tube defining a channel extending from the proximal end of the tube to an aperture at the distal end and wherein the expansible member is housed within the channel prior to deployment within the anatomical lumen; wherein the delivery state is a compressed state; wherein the instrument extends proximally out of the channel and is configured to control axial movement of the expansible member relative to the tube; wherein the expansible member has a proximal end and a distal end, and markers are positioned proximate at least one of the distal and proximal ends of the expansible member; wherein the expansible member has a tapered proximal end to facilitate releasable engagement with a distal end of the instrument; wherein the expansible member includes a protrusion at the proximal end to facilitate engagement between the instrument and the expansible member; wherein the expansible member comprises a material that exhibits an expansion/compression size ratio of approximately 10:1; wherein the expansible member comprises poly-vinyl alcohol (PVA); wherein the expansible member comprises a sponge; wherein the expansible member defines holes formed therein for passing irrigation therethrough in the expanded state; wherein the expansible member comprises a material less susceptible to laser-energy induced damage than alloys of nickel/titanium, copper, cobalt, vanadium, chromium, and iron; wherein the instrument comprises grasping forceps; wherein the expansible member expands to the contours of the anatomical lumen in the expanded, deployed state; wherein the expansible member expands to fill a cross-sectional area of an anatomical lumen in the expanded, deployed state; wherein the expansible member, in the expanded, deployed state, exhibits a sufficient strength to prevent movement of the material past the expansible member within the anatomical lumen; wherein the instrument comprises a treatment device having a collapsible basket; wherein the expansible member expands to the deployed state due to the presence of fluid; and wherein the expansible member is configured in the expanded, deployed state to be unconnected to any structure within the anatomical lumen that extends proximally of the material.

Another embodiment of the invention is directed to a method for immobilizing material in a body including inserting an expansible member into an anatomical lumen of the body, the expansible member having a delivery state, an expanded state, and a proximal end detachably engaged with a distal end of an instrument. The method further includes positioning the instrument to deploy the expansible member such that the expansible member transforms from the delivery state to the expanded state at a treatment site within the anatomical lumen. The method includes detaching the instrument from the expansible member.

In various embodiments, the method may include one or more of the following additional features: wherein inserting an expansible member includes providing an elongated flexible tube including a distal end and a proximal end, the tube defining a channel extending from the proximal end to an aperture at the distal end and wherein the expansible member is housed within the channel prior to deployment at a treatment site; wherein positioning the instrument to deploy the expansible member includes moving the instrument relative to the tube to control axial movement of the expansible member beyond the channel; wherein the expansible member comprises a material that expands to the expanded state when unrestrained and in the presence of fluid; wherein the expansible member is deployed distally beyond the material to be immobilized such that the expansible member at least partially occludes the anatomical lumen; performing a lithotripsy procedure on the material; irrigating the lumen of the body; retrieving the immobilized material by reattaching the instrument with the proximal end of the expansible member and proximally pulling the expansible member through the anatomical lumen with the expansible member in the expanded state; wherein the anatomical lumen includes an interior surface and the expansible member expands to contact the interior surface of the anatomical lumen; wherein the expansible member comprises a material that exhibits an expansion/compression size ratio of approximately 10:1; wherein the expansible member comprises poly-vinyl alcohol (PVA); wherein the expansible member comprises a sponge; wherein the expansible member defines holes formed therein for passing irrigation therethrough in the expanded state; wherein the expansible member comprises a material less susceptible to laser-energy induced damage than alloys of nickel/titanium, copper, cobalt, vanadium, chromium, and iron; wherein the expansible member has a proximal end and a distal end, and markers are positioned proximate the distal and proximal ends of the expansible member; wherein positioning further includes visualizing the position of the markers through a medical imaging device; retrieving the immobilized material by engaging fragmented immobilized material with the instrument; wherein the expansible member includes a protrusion at the proximal end to facilitate engagement between the instrument and the expansible member; wherein deployment of the expansible member results in expansion of the expansible member to the contours of an anatomical lumen in the deployed state; wherein the expansible member, in the deployed state, exhibits a sufficient strength to prevent movement of material past the expansible member within an anatomical lumen; and wherein, after the detaching the instrument, the expansible member in the expanded state is unconnected to any structure within the anatomical lumen that extends proximally of the material.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1 illustrates a medical immobilization/retrieval assembly introduced within a proximal portion of an endoscope, according to an embodiment of the invention.

FIG. 2A is a partial side cross-sectional view of a distal portion of a medical immobilization/retrieval assembly in an undeployed position, according to an embodiment of the invention.

FIG. 2B is a partial side cross-sectional view of the medical immobilization/retrieval assembly of FIG. 2A in a partially deployed position, according to an embodiment of the invention.

FIG. 3 illustrates the medical immobilization/retrieval assembly of FIG. 1 having an expansible member deployed beyond a kidney stone in a ureter, according to an embodiment of the invention.

FIG. 4 illustrates the retraction of a retrieval instrument after deployment of the immobilization/retrieval assembly, according to an embodiment of the invention.

FIG. 5 illustrates a medical immobilization/retrieval assembly having an expansible member deployed beyond a kidney stone in the ureter and a lithotripsy device positioned proximal to the kidney stone, according to an embodiment of the invention.

FIG. 6 illustrates the retrieval of a kidney stone after fragmentation by a medical lithotripsy device, according to an embodiment of the invention.

FIG. 7 illustrates a medical immobilization device in a partially deployed position, according to another embodiment of the invention.

FIG. 8 illustrates a medical immobilization/retrieval assembly deployed beyond a kidney stone in a ureter, according to another embodiment of the invention.

FIG. 9 illustrates the retraction of a retrieval instrument after deployment of the immobilization/retrieval assembly, according to another embodiment of the invention.

### DESCRIPTION OF THE EMBODIMENTS

Reference will now be made in detail to the present exemplary embodiments of the invention illustrated in the accompanying drawings. Wherever possible, the same reference numbers will be used throughout the drawings to refer to the same or like parts. The drawing figures of this application are intended to provide a general understanding of the working elements of the underlying system. Accordingly, unless explicitly stated, the figures do not represent a literal depiction of proportional dimensions or the precise locations for the illustrated inter-related components.

Endoscopes are used for non-invasively viewing an internal body cavity of a patient with imaging structure housed therein. In addition to viewing and lighting an internal body portion, endoscopes also serve to position treatment instruments within a particular body portion for performing various surgical and diagnostic functions within a desired internal body portion. For purposes of this disclosure, “treatment device” or “treatment instrument” includes, for example, any medical device advanced through a working channel of an endoscope and for use during an endoscopic procedure. Exemplary treatment instruments include, but are not limited to, guide wires, cutting or grasping forceps, biopsy devices, snare loops, injection needles, cutting blades, scissors, retractable baskets, retrieval devices, ablation and/or electrophysiology catheters, stent placement devices, surgical stapling devices, lithotripsy or other fragmentation devices, and balloon catheters.

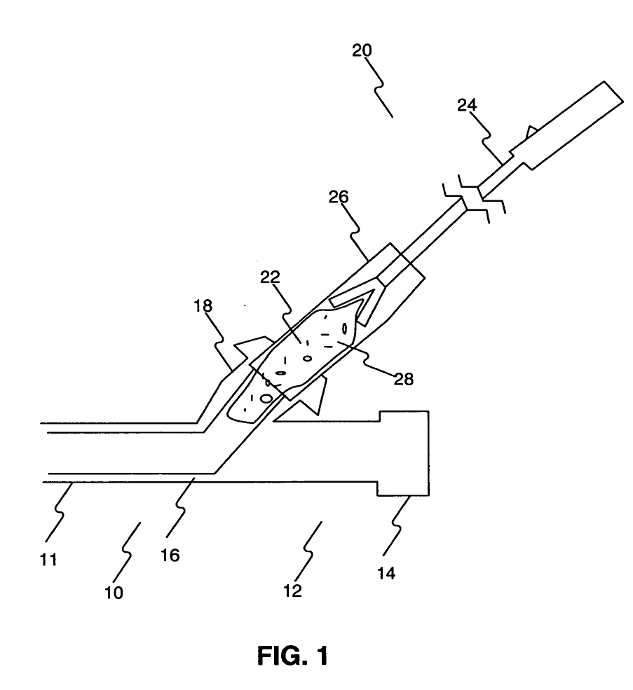

FIG. 1 illustrates an endoscope 10 including a flexible outer tube 11 having a proximal portion 12 and a distal portion (not shown). For purposes of this disclosure, “distal” refers to the end further from the device operator during use and “proximal” refers to the end closer to the device operator during use. As is known in the art, conventional endoscopes often terminate proximally at a handle 14 configured for manipulation by an operator. In addition, conventional endoscopes further include a working channel 16 therein extending from an access port 18 at a proximal end of the endoscope and leading to an exit aperture at a distal end of the endoscope. Treatment instruments, therefore, can be deployed at desired treatment area within an anatomical lumen of a patient by insertion through the access port 18 and extension through the working channel 16.

FIG. 1 further depicts a medical immobilization/retrieval assembly 20 introduced within the access port 18 of an endoscope 10. In one embodiment, the immobilization/retrieval assembly 20 includes an immobilization device 22, a retrieval instrument, such as, for example, grasping forceps 24, and an introducer sleeve 26. Introducer sleeve 26 is aligned with the access port 18 such that the assembly 20 can be extended within the working channel 16 of the endoscope 10 for selective placement at a treatment site.

The immobilization device 22 is initially housed within the introducer sleeve 26. An operator can selectively deploy the assembly 20 upon distal movement of the assembly relative to both the introducer sleeve 26 and the working channel 16. In one embodiment, the immobilization device comprises an expansible member 28. The expansible member 28 is constrained in a collapsed state by introducer sleeve 26 and is movable relative to the flexible tube 11 within channel 16.

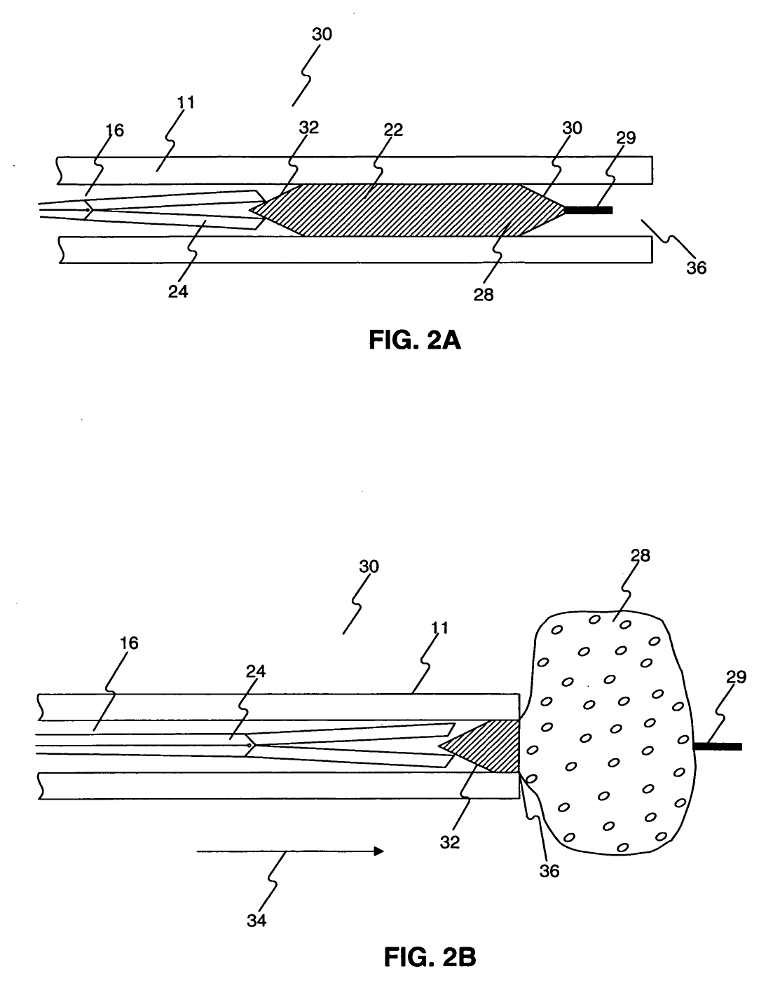

FIG. 2A is a partial side cross-sectional view illustrating a distal portion 30 of endoscope 10. As seen in FIG. 2A, forceps 24 and expansible member 28 are further extended within channel 16 of endoscope 10. Expansible member 28 is depicted in an undeployed compressed configuration. Marker bands 29, such as radiopaque markers or other suitable markers, can be attached to the expansible member 28 at the distal and proximal ends of the expansible member 28. The expansible member 28 is constrained in a collapsed state by channel 16 and is movable relative to the flexible tube 11 within channel 16. The expansible member 28 may further include a tapered distal portion 30 and a tapered proximal portion 32 to facilitate the movement of the expansible member 28 in forward deployment from within the channel 16 and in retraction back within channel 16. In particular, the proximal tapered portion 32 may be configured for engagement by the jaws of grasping forceps 24. For example, grasping forceps 24 could be manipulated by an operator at the proximal end of the endoscope 10 in order to control deployment of the assembly at a desired treatment location.

As seen in FIG. 2B, forceps 24 and expansible member 28 are configured for forward movement relative to the channel 16, in the direction of arrow 34. The tube 11 and expansible member 28 are movable relative to each other in order to achieve a first, compressed, collapsed state (FIG. 2A) in which the expansible member 28 is compressed within the internal working channel 16 of the flexible tube 11 and a second state (FIG. 2B) in which the expansible member 28 extends from the distal opening 36 of the channel 16 and expands to greater size.

The expansible member 28 may consist of a material that expands in the presence of fluid when unconstrained by the channel 16. For example, expansible member 28 can be formed of a poly-vinyl alcohol (PVA) sponge and may have an expansion ratio of approximately 10:1. A poly-vinyl alcohol having other suitable expansion ratios may be used. In addition to poly-vinyl alcohol, the expansible member 28 can be formed of any biocompatible material suitable for placement within an anatomical lumen of a patient's body that exhibits a high expansion/compression ratio. Suitable alternative materials may include, but are not limited to, soft polyurethane or polyethylene foam. Although poly-vinyl alcohol sponge is disclosed, any biocompatible materials that have a relatively high compression ratio and can be shaped and gripped by grasping forceps (or a basket) can be used. Instead of an expanding material like poly-vinyl alcohol, alternative materials may include, but are not limited to, compressible and/or flexible materials, such as, for example, silicone hydrogel, silicone-acrylate, fluorosilicone acrylate, polymethyl methacrylate (PMMA), or USAN-filicon materials. These compressible and/or flexible materials could be provided at a treatment site by being folded, wrung, necked, or otherwise reduced in overall size for initial containment within an introducer sleeve or an internal working channel of a medical delivery device.

The use of a poly-vinyl alcohol material, or the like, is advantageous in that such material is less susceptible to laser-energy induced damage than other immobilization/retrieval device materials, such as traditional metals and alloys thereof. In addition, since the poly-vinyl alcohol material absorbs water, the resulting deployed structure of the expansible member 28 is further resistant to damage from laser energy.

FIG. 2B illustrates a distal portion of expansible member 28 being deployed beyond the distal opening 36 of channel 16. In the presence of liquid, such as, for example, upon deployment within the ureter of a patient, the unconstrained portion of the expansible member 28 will absorb fluid and expand according to the physical properties of the expansible member. As noted above, the immobilization device 22 is configured for movement relative to the channel 16. Any such relative movement may be controlled by a user at a proximal end of the endoscope 10 through manipulation of grasping forceps 24.

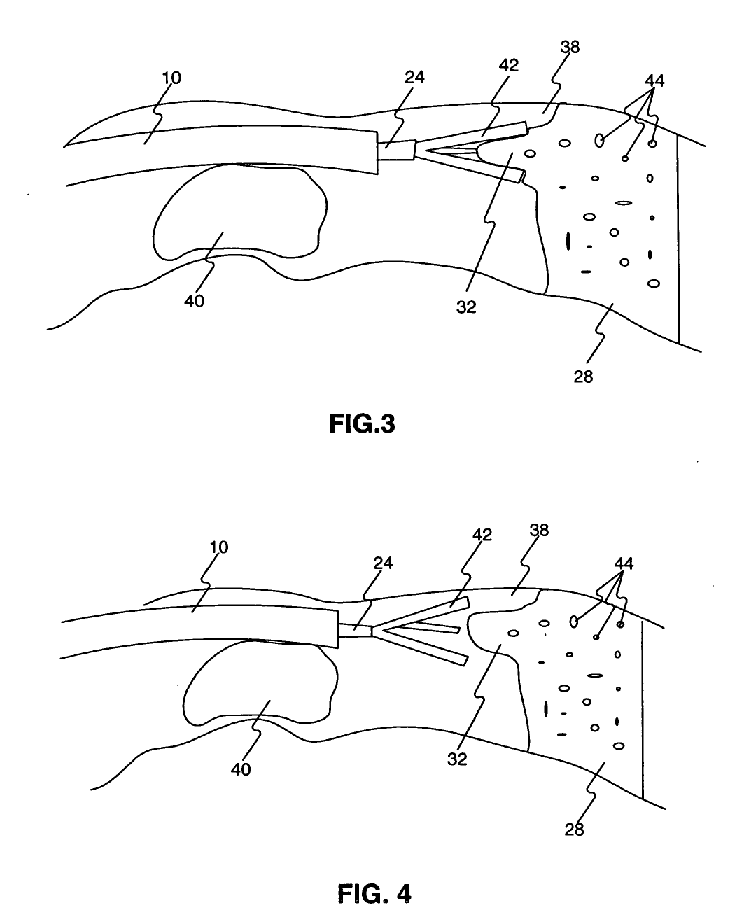

FIGS. 3-9 illustrate systems and methods for retrieving, immobilizing, and/or preventing migration of objects in anatomical lumens during a medical procedure. Referring to FIG. 3, endoscope 10 may be positioned at an internal treatment site, for example, such as within a patient's ureter 38. The positioning may be performed by any suitable method known in the art, and may include known imaging and viewing techniques. The distal end of endoscope 10 is positioned distally beyond a kidney stone 40, for example. As seen in FIG. 3, expansible member 28 is then deployed completely beyond the working channel 16 of endoscope 10, thereby absorbing fluid and achieving the illustrated expanded state. As noted above, grasping forceps 24 can be used to control deployment of the expansible member 28. The jaws 42 of grasping forceps 24 can be used to engage a proximal tapered portion 32 of the expansible member 28 in order to selectively manipulate the proper placement of the deployed immobilization device 22.

The tapered proximal portion 32 of the expansible member 28 can be formed to exhibit a tapered shape, or any other protruding shape, configured to facilitate grasping and engagement by forceps 24. For example, the proximal portion of expansible member 28 can be formed to maintain the particular predetermined protruding shape, even in the presence of fluid, in order to facilitate engagement by forceps 24 for controlled deployment, positioning, and retraction of the expansible member 28. In addition, the proximal portion of expansible member 28 can be formed to include multiple protrusions to facilitate grasping and engagement by forceps 24. FIG. 3 further illustrates that in an expanded state, expansible member 28 fills a portion of ureter 38 while still adjusting to comply with contours and turns that may exist along the lumen within which the expansible member is deployed.

In embodiments where the expansible member consists of a PVA sponge material, the expansible member 28 is hydrated when deployed within a patient's anatomical body lumen, thereby expanding to expose pores 44. The pore size may be varied as appropriate depending on the desired rigidity of the sponge material. Holes defining apertures (not shown), separate from the pores 44, can be manufactured in the sponge. The holes can be used for the passage of irrigation and to provide a smaller profile of the sponge in the compressed state. The hole diameter would ideally be less than 4 mm to prevent a larger stone from being imbedded in the sponge or migrating past the sponge during deployment.

Depending on the size of the targeted stone 40, expansible member 28 can be used in combination with grasping forceps 24 as a tool to move a stone 40 proximally along a ureter 38. In the expanded state of FIG. 3, expansible member 28 can be moved proximally along the ureter 38 to sweep stone 40 in the proximal direction. Upon the continued proximal movement of expansible member 28, stone 40 may be swept and repositioned to a new location more accessible for retrieval and removal, or swept from the ureter altogether. The material of expansible member 28, such as poly-vinyl alcohol, for example, is selected to be soft enough when expanded not to damage the surrounding tissue during movement and yet rigid enough to sweep or immobilize a stone 40.

FIG. 4 illustrates the retraction of a retrieval instrument, such as, for example, grasping forceps 24 after deployment of the expansible member 28. As seen in FIG. 4, an operator can control the actuation of the jaws 42 of grasping forceps 24 in order to release the expansible member 28 after proper placement and deployment. Grasping forceps 24 can then be retracted within channel 16 of endoscope 10. Grasping forceps 24 can then be completely backed out of the channel 16 in order to allow deployment of additional treatment instruments within channel 16 as may be appropriate depending on the particular medical procedure.

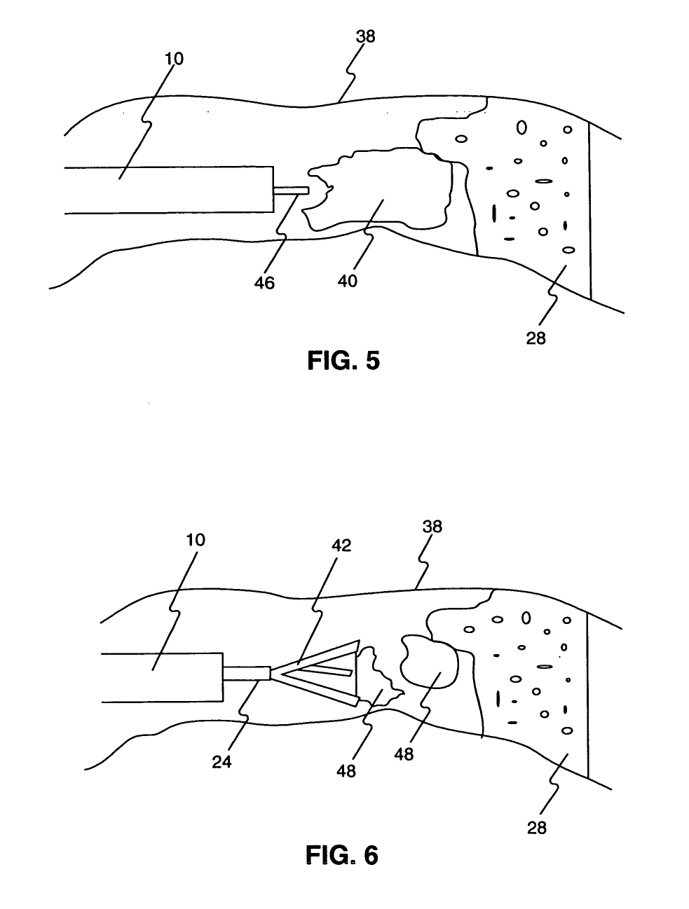

For example, FIG. 5 illustrates the deployment of a lithotriptor 46 beyond the working channel 16 of endoscope 10. Where a concretion, such as kidney stone 40, is too large to be extracted without fragmentation, a lithotriptor 46 can be advanced through the working channel 16 of an endoscope 10 in order to perform the fragmentation. Lithotriptor 46 may be, for example, a laser fiber for directing laser energy at kidney stone 40 in order to break down the concretion into smaller pieces to facilitate retrieval or normal passage through the bladder.

One advantage of the embodiment of the immobilization/retrieval assembly 20, illustrated in FIGS. 1-5, is the detachment of expansible member 28 from the introducing device, in this case grasping forceps 24. As noted above, in past devices where a backstop was deployed distally beyond a kidney stone, the proximal portion of the backstop (which necessarily extends outside the patient) congests and obstructs the free movement of an associated lithotriptor device. Such congestion is further exacerbated where the treatment procedure requires the use of an endoscope at the working area for lighting and imaging the treatment area. The resulting congestion at the treatment site hinders the free movement of various components placed at the working area during a treatment procedure.

In the embodiments of FIGS. 1-5, expansible member is completely detached from any introducing instruments. Therefore, the working region of the ureter 38 no longer houses a proximal portion of the backstop. The detachable arrangement of assembly 20 therefore facilitates the free movement of an associated lithotriptor (or any other treatment device required by the procedure) by allowing a less congested internal working area at the desired treatment site. The expanded working area created by the detachable arrangement of assembly 20 thereby allows for more precise control and movement for treatment devices, such as a lithotripter 46. Accordingly, safer treatment procedures are realized.

Another advantage of the embodiment of the immobilization/retrieval assembly 20 is the benefits afforded by the material of construction for immobilization device 22. As noted above, past backstop and retrieval devices were formed of materials susceptible to damage from laser energy. As a result of damage from laser energy, broken or roughened portions of the past devices led to trauma of the delicate internal lining of the patient's ureter. In addition, further damage to the patient occurred where misdirected laser energy was improperly absorbed by the retrieval device, thereby heating the retrieval device (and patient tissue) to unintended dangerous levels.

As an alternative procedure, the expansible member may be positioned along or around a portion of the targeted material. This arrangement serves to protect healthy tissue from incidental exposure to the energy of the lithotriptor in addition to, or as an alternative to, functioning as a backstop or immobilization device.

In the embodiments of FIGS. 1-5, immobilization device can be formed of a material less susceptible to damage from laser energy. For example, the use of a poly-vinyl alcohol material, or the like, is advantageous in that such material absorbs water thereby exhibiting resistance to damage from laser energy. In addition, such a material should exhibit an ability to absorb relatively large amounts of water by weight as well as exhibit a relatively low specific heat. These characteristics, especially in combination with irrigation during a medical procedure, provide the benefits of a material less susceptible to damage from laser energy.

As seen in FIG. 6, after a lithotripsy procedure is completed, kidney stone 40 is fragmented into multiple smaller stones 48. During the lithotripsy procedure, the expansible member 28 serves as a backstop to prevent migration of the smaller stones 48, thereby preventing complications resulting from the potential migration of stones 48 back into a patient's kidneys. After the fragmentation of stone 40, the lithotriptor 46 is withdrawn and the expansible member 28 can then be pulled proximally along the ureter 38 to sweep the remaining smaller stones 48 toward the bladder to be voided or repositioned to facilitate retrieval by an additional retrieval device.

For example, FIG. 6 depicts the redeployment of grasping forceps 24 within the patient's ureter 38 in the location of the smaller stones 48. As illustrated, the jaws 42 of grasping forceps can be manipulated to capture the stone fragments 48 for removal from the patient. After a successful treatment procedure, grasping forceps 24 can be used to retract the expansible member 28 back within the working channel of the endoscope 10. The expansible member 28 is not, however, required to be removed from the treatment site through the endoscope 10 and alternative removal methods will be apparent to one having ordinary skill in the art.

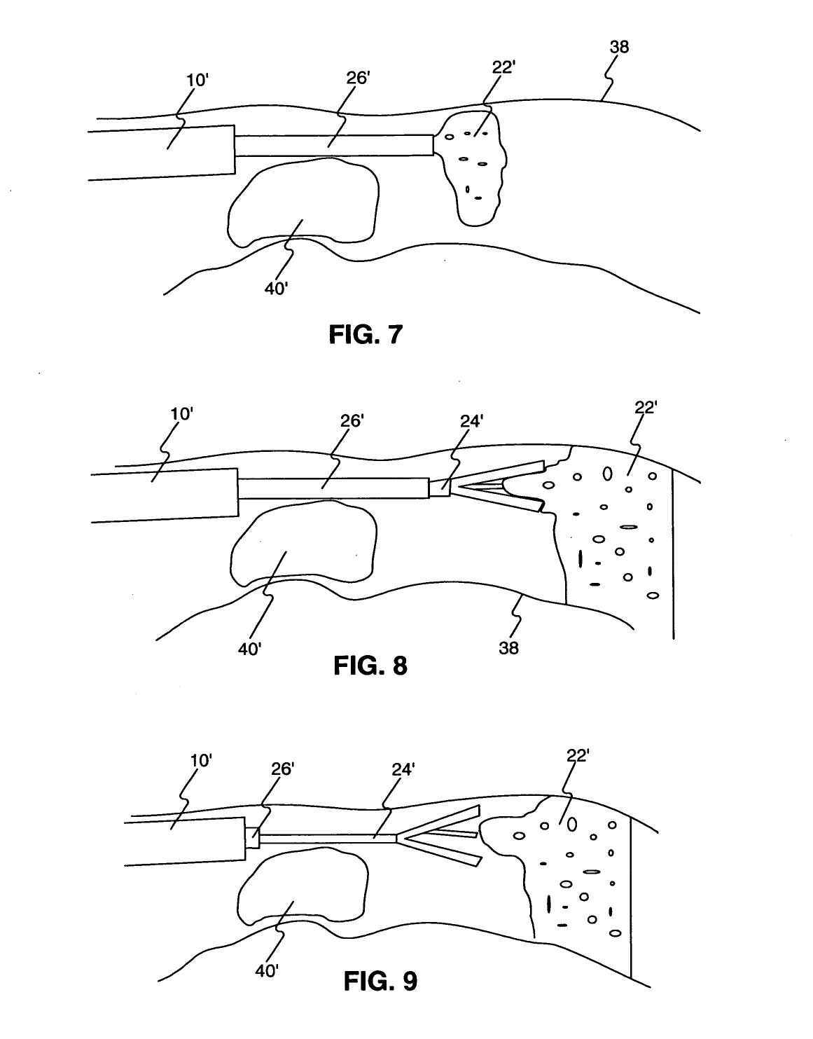

FIGS. 7-9 illustrate an additional embodiment of the present invention directed to a endoscope 10′ including an extended introducer sleeve 26′ for use with immobilization device 22′. Sleeve 26′ is longer in comparison to previously described sleeve 26 and is sized (i.e. has a sufficiently small diameter) for extending within a working channel of endoscope 10′. The introducer sleeve 26′ could act as a delivery sheath for an expansible or compressible material housed therein forming an immobilization device 22′. The sleeve 26′ could be formed of a polytetrafluoroethylene (PTFE), polyimide, nylon, or other biocompatible polymer materials exhibiting column strength and flexibility. In addition, the sheath could be braided to add additional kink resistance.

As seen in FIGS. 7-9, introducer sleeve 26′ can be extended beyond the distal end of the endoscope 10′ in order to more precisely control the location of backstop deployment. As depicted in FIG. 7, some stones, such as kidney stone 40′, encountered within a patient's ureter 38 will be of a size that prevents the placement of endoscope 10 distally beyond the stone 40′. In such situations, the smaller diameter of sleeve 26′, relative to the endoscope 10, allows for the deployment of a backstop 22′ distally beyond the targeted stone 40′.

In embodiments of the invention utilizing sleeve 26′, the grasping forceps 24′ and the backstop 22′ must be of a size configured for introduction and deployment through the reduced diameter of sleeve 26′. Once deployed, as seen in FIG. 9, the grasping forceps 24′ and sleeve 26′ can be selectively retracted proximally in order to traverse the congested location of the stone 40′. After this retraction, a fragmentation procedure can be performed using a lithotriptor as described above.

While this specification makes reference to endoscope devices, the invention is not intended to be so limited. Accordingly, the elements described in this application may be used with any other medical device requiring, or even benefiting from, an immobilization/retrieval assembly. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A medical device, comprising:
an expansible member having a proximal end, a delivery state, and an expanded, deployed state;
an instrument detachably engaged with the proximal end of the expansible member to deploy the expansible member within an anatomical lumen beyond material to be retrieved; and
wherein the expansible member in the expanded state is configured to inhibit movement of the material within the anatomical lumen.

2. The medical device of claim 1, further comprising an elongated flexible tube including a distal end and a proximal end, the tube defining a channel extending from the proximal end of the tube to an aperture at the distal end and wherein the expansible member is housed within the channel prior to deployment within the anatomical.

3. The medical device of claim 1, wherein the delivery state is a compressed state.

4. The medical device of claim 2, wherein the instrument extends proximally out of the channel and is configured to control axial movement of the expansible member relative to the tube.

5. The medical device of claim 1, wherein the expansible member has a proximal end and a distal end, and markers are positioned proximate at least one of the distal and proximal ends of the expansible member.

6. The medical device of claim 1, wherein the expansible member has a tapered proximal end to facilitate releasable engagement with a distal end of the instrument.

7. The medical device of claim 1, wherein the expansible member includes a protrusion at the proximal end to facilitate engagement between the instrument and the expansible member.

8. The medical device of claim 1, wherein the expansible member comprises a material that exhibits an expansion/compression size ratio of approximately 10:1.

9. The medical device of claim 1, wherein the expansible member comprises poly-vinyl alcohol (PVA).

10. The medical device of claim 1, wherein the expansible member comprises a sponge.

11. The medical device of claim 1, wherein the expansible member defines holes formed therein for passing irrigation therethrough in the expanded state.

12. The medical device of claim 1, wherein the expansible member comprises a material less susceptible to laser-energy induced damage than alloys of nickel/titanium, copper, cobalt, vanadium, chromium, and iron.

13. The medical device of claim 1, wherein the instrument comprises grasping forceps.

14. The medical device of claim 1, wherein the expansible member expands to the contours of the anatomical lumen in the expanded, deployed state.

15. The medical device of claim 1, wherein the expansible member expands to fill a cross-sectional area of an anatomical lumen in the expanded, deployed state.

16. The medical device of claim 1, wherein the expansible member, in the expanded, deployed state, exhibits a sufficient strength to prevent movement of the material past the expansible member within the anatomical lumen.

17. The medical device of claim 1, wherein the instrument comprises a treatment device having a collapsible basket.

18. The medical device of claim 1, wherein the expansible member expands to the deployed state due to the presence of fluid.

19. The medical device of claim 1, wherein the expansible member is configured in the expanded, deployed state to be unconnected to any structure within the anatomical lumen that extends proximally of the material.

20. A method for immobilizing material in a body comprising:
inserting an expansible member into an anatomical lumen of the body, the expansible member having a delivery state, an expanded state, and a proximal end detachably engaged with a distal end of an instrument;
positioning the instrument to deploy the expansible member such that the expansible member transforms from the delivery state to the expanded state at a treatment site within the anatomical lumen; and
detaching the instrument from the expansible member.

21. The method of claim 20, wherein inserting an expansible member includes providing an elongated flexible tube including a distal end and a proximal end, the tube defining a channel extending from the proximal end of the tube to an aperture at the distal end, and wherein the expansible member is housed within the channel prior to deployment at the treatment site.

22. The method of claim 21, wherein positioning the instrument to deploy the expansible member includes moving the instrument relative to the tube to control axial movement of the expansible member beyond the channel.

23. The method of claim 20, wherein the expansible member comprises a material that expands to the expanded state when unrestrained and in the presence of fluid.

24. The method of claim 20, wherein the expansible member is deployed distally beyond the material to be immobilized such that the expansible member at least partially occludes the anatomical lumen.

25. The method of claim 24, further comprising performing a lithotripsy procedure on the material.

26. The method of claim 20, further comprising irrigating the anatomical lumen.

27. The method of claim 24, further comprising retrieving the immobilized material by reattaching the instrument with the proximal end of the expansible member and proximally pulling the expansible member through the anatomical lumen.

28. The method of claim 20, wherein the anatomical lumen includes an interior surface and the expansible member expands to contact the interior surface of the anatomical lumen.

29. The method of claim 20, wherein the expansible member comprises a material that exhibits an expansion/compression size ratio of approximately 10:1.

30. The method of claim 20, wherein the expansible member comprises poly-vinyl alcohol (PVA).

31. The method of claim 20, wherein the expansible member comprises a sponge.

32. The method of claim 20, wherein the expansible member defines holes formed therein for passing irrigation therethrough in the expanded state.

33. The method of claim 20, wherein the expansible member comprises a material less susceptible to laser-energy induced damage than alloys of nickel/titanium, copper, cobalt, vanadium, chromium, and iron.

34. The method of claim 20, wherein the expansible member has a proximal end and a distal end, and markers are positioned proximate the distal and proximal ends of the expansible member.

35. The method of claim 34, wherein positioning further includes visualizing the position of the markers through a medical imaging device.

36. The method of claim 25, further comprising retrieving the immobilized material by engaging fragmented immobilized material with the instrument.

37. The method of claim 20, wherein the expansible member includes a protrusion at the proximal end to facilitate engagement between the instrument and the expansible member.

38. The method of claim 20, wherein deployment of the expansible member results in expansion of the expansible member to the contours of the anatomical lumen.

39. The method of claim 20, wherein deployment of the expansible member results in expansion of the expansible member to fill a cross-sectional area of the anatomical lumen.

40. The method of claim 20, wherein the expansible member, in the expanded state, exhibits a sufficient strength to prevent movement of material past the expansible member within the anatomical lumen.

41. The method of claim 20, wherein, after the detaching the instrument, the expansible member in the expanded state is unconnected to any structure within the anatomical lumen that extends proximally of the material.
